(19) United States
(12) Patent Application Publication
(10) Pub. No.: US 2008/0234822 A1
(43) Pub. Date: Sep. 25, 2008

(54) Method and Apparatus for Stabilization and Fusion of Adjacent Bone Segments

(75) Inventors: Amit Govil, San Diego, CA (US); Jennifer Faleris, Gainesville, FL (US); Adam Poniatowski, Gainesville, FL (US); John McDougall, Gainesville, FL (US)

Correspondence Address: THOMAS, KAYDEN, HORSTEMEYER & RISLEY, LLP, 600 GALLERIA PARKWAY, S.E., STE 1500, ATLANTA, GA, 30339-5994, US

(73) Assignee: TUTOGEN MEDICAL, U.S., INC., Alachua, FL (US)

(21) Appl. No.: 11/627,611

(22) Filed: Jan. 26, 2007

(51) Int. Cl. A61F 2/44; A61B 17/58

(52) U.S. Cl. 623/17.16

(57) ABSTRACT

Disclosed are systems and methods for stabilization and fusion of adjacent bone segments. Also disclosed are methods for manufacturing a surgical bone implant. Surgical bone implants constructed in accordance with the invention receive improved structural stability and accurate sizing, particularly under expansion and shrinkage caused by hydration and dehydration of the implant.

### FIELD OF THE INVENTION

The present invention generally relates to surgical orthopedic implants, and, more specifically, to systems and methods for stabilization and fusion of adjacent bone segments.

### BACKGROUND OF THE INVENTION

The human spine is composed of a column of thirty-three bones, called vertebra, and their adjoining structures. The twenty-four vertebrae nearest the head are separate bones capable of individual movement and are generally connected by anterior and posterior longitudinal ligaments and by discs of fibrocartilage, called intervertebral discs, positioned between opposing faces of adjacent vertebrae. The twenty-four vertebrae are commonly referenced in three sections. The cervical spine, closest to the head and often referenced as the “neck,” comprises the first seven vertebrae of the spine. The thoracic spine and the lumbar spine are below the cervical spine. The lumbar spine comprises the five vertebrae situated between the thoracic region and the sacrum of the spine. Each of the vertebrae includes a vertebral body and a dorsal arch, which enclose an opening, called the vertebral foramen, through which the spinal cord and the spinal nerve pass. The remaining nine vertebrae below the lumbar spine are fused to form the sacrum and the coccyx and are incapable of individual movement.

Lumbar interbody fusion procedures are a set of various surgical procedures designed to treat painful or otherwise abnormal conditions in or emanating from the various regions of the spine. Such a procedure may generally involve the fusing of two adjacent vertebrae in the spine to restrict or eliminate their movement relative to each other. The procedure can be administered in response to numerous conditions involving a defect or instability of the above mentioned regions of the spine. An exemplary condition of a patient's spine that may create a need for such a surgical procedure can include a degenerative disc disease, which can involve problems with one or more intervertebral discs. An intervertebral disc is designed to absorb pressure and keep a patient's spine flexible by cushioning stresses and movement of the spine. An intervertebral disc may lose its ability to act as a cushion between vertebrae of the spine, which can cause two vertebrae above and below the disc to move closer together. This may cause lower body pain as well as other issues including but not limited to a shifting of facet joints located at the back of the spine.

Another exemplary condition for with spinal fusion procedures may offer relief to patients is a spinal instability resulting in excess movement of one or more vertebrae causing an irritation or pinching of the nerves in the spine, which can lead to lower body pain as well as muscle spasms of the muscles in the surrounding area. A spinal instability of this nature can cause a degeneration of the spine in the area of the instability. Additionally, spinal fusion may be required to correct conditions causing an abnormal curvature of the spine, which can include but are not limited to scoliosis and kyphosis. Spondylolisthesis, which is a condition of a patient's spine where one vertebra slips relative to another vertebra above or below, may also require correction with an anterior lumbar interbody fusion procedure to prevent further slippage of the vertebra. It should be noted that the above is not an exhaustive list of conditions or injuries to a patient's spine which may cause a surgeon to choose to perform a spinal procedure, as other spinal conditions and injuries may necessitate or motivate such a procedure, such as but not limited to injuries resulting from spinal trauma.

Generally, a spinal fusion procedure involves a surgical approach to a patient's spine, upon which a surgeon may then remove at least a portion of an intervertebral disc located between two vertebrae to prepare the area for the insertion of a bone graft or spinal implant. The disc can be chosen for removal by the surgeon if it is in a degenerative state or causing spinal instability in the lumbar region of the spine. A bone graft or spinal implant or a combination of both can then be inserted into or around the area of removed intervertebral disc to facilitate the fusion of two adjacent vertebrae. As is known in the art, such bone grafts or spinal implants can be machined from a collection of harvested bone fragments that are fused and machined to form a shape appropriate for insertion into or near a patient's spine. Such implants are generally made of cortical, cancellous, corticocancellous or a combination of all three aforementioned types of bone material. Implants may also include osteoinductive and/or osteoconductive materials to facilitate fusion and/or new bone growth in or around the area of implant insertion, as is known in the art.

While the design and materials used within certain implants used in various spinal fusion procedures may be known, it is also known in the art that because of the methods of manufacturing currently practiced in the art, the stability of various spinal implants used in spinal fusion procedures can be less than desirable. This can be caused by the manufacturing, sterilization, and storage processes that are known in the art. As is known, spinal implants may be made from a plurality of bone fragments that are formed into a larger fragment that is subsequently machined into a shape appropriate for a spinal implant. After machining into an appropriate shape, implants made of bone material may be dehydrated for storage and/or transportation. It is known that a dehydrated implant constructed from cancellous or cortical bone fragments may also be sterilized prior to packaging.

A dehydrated implant must be rehydrated prior to use by a surgeon, and it is known that these steps of dehydration and rehydration may result in a physical weakening or breakdown of an implant. One reason for the lessening of the physical stability of the implant is warping cause by the shrinkage and expansion of the implant that occurs due to dehydration and subsequent rehydration of an implant containing bone fragments. Another reason for the lack of stability is that implants are often comprised of a plurality of bone fragments that may expand and shrink as they are hydrated or dehydrated in an unpredictable manner. This unpredictability is known as the direction and level to which expansion and shrinkage occurs. Bone implants known in the prior art may be comprised of bone fragments where the osteonal direction of the fragments is oriented in various directions. Because of this structure, the bone fragments or pulverized bone may shrink or expand is various directions. Such unpredictable expansion and shrinkage may lead to a fracturing or breakdown of a bone implant containing bone material because of warping. Such unpredictable expansion and shrinkage may also lead to difficulties in achieving proper sizing of a surgical bone implant, particularly in a vertical direction when an implant is positioned between vertebrae in a patient's spine.

Another cause of unpredictable shrinkage or expansion is due to the dehydration process of a bone implant prior to storage or sterilization. Some dehydration processes may cause lipids to be removed from bone fragments making up an implant. As is known, considering the brittle nature of bone implants, some lipids within a bone fragment may assist the physical stability of the fragment and therefore promote the physical stability of a bone implant containing a plurality of bone fragments. Hence, there is a need for stronger and more physically stable bone implants made of single source harvested bone fragments or pulverized bone, particularly because spinal implants must possess sufficient strength and rigidity because of the location of their use in a patient's spine. In light of the foregoing, there are deficiencies in the prior art that have been heretofore unaddressed.

### SUMMARY OF THE INVENTION

The present disclosure is related to methods and apparatuses for stabilization and fusion of adjacent bone segments. Briefly described, in architecture, one embodiment of the system, among others, can be implemented as follows: a first bone fragment machined for interlocking with a second bone fragment. The first bone fragment is machined such that the osteonal direction is parallel to a direction of insertion with the second bone fragment. The first bone fragment and the second bone fragment are configured to form an interlocking assembly by dehydrating the first bone fragment, interlocking the first bone fragment and the second bone fragment to form an assembly, and further dehydrating the assembly.

One embodiment of a method includes a method of manufacturing a surgical bone implant. The method includes machining a first bone fragment for engagement with a second bone fragment, where the first bone fragment configured to interlock with the second bone fragment in at least one joint and the first bone fragment is machined as a male portion of the at least one joint and the second bone fragment machined as a female portion of the at least one joint. The method also includes machining at least one of the first bone fragment and the second bone fragment with at an osteonal direction parallel to a direction of insertion with the second bone fragment. The method further includes at least partially dehydrating the first bone fragment and interlocking the first bone fragment with the second bone fragment. The method finally includes at least partially dehydrating the interlocking first bone fragment and second bone fragment.

### BRIEF DESCRIPTION OF THE DRAWINGS

Many aspects of the disclosure can be better understood with reference to the following drawings. The components in the drawings are not necessarily to scale, emphasis instead being placed upon clearly illustrating the principles of the present disclosure. Moreover, in the drawings, like reference numerals designate corresponding parts throughout the several views.

FIG. 1 depicts a perspective view of a surgical bone implant in accordance with the disclosure.

FIG. 2 depicts a perspective view of a surgical bone implant in accordance with the disclosure.

FIG. 3 depicts a perspective view of a surgical bone implant in accordance with the disclosure.

FIG. 4 depicts a perspective view of a surgical bone implant in accordance with the disclosure.

FIG. 5 depicts a perspective view of a surgical bone implant in accordance with the disclosure.

FIG. 6 depicts a perspective view of a surgical bone implant in accordance with the disclosure.

FIG. 7 depicts a perspective view of a surgical bone implant in accordance with the disclosure.

FIG. 8 depicts a flow diagram of a method in accordance with an embodiment of the disclosure.

FIG. 9 depicts a flow diagram of a method in accordance with an embodiment of the disclosure.

### DETAILED DESCRIPTION OF THE DRAWINGS

For the purposes of promoting and understanding of the present invention, reference will now be made to various embodiments and illustrations in the drawings. While the disclosure will be provided in connection with the drawings, there is no intent to limit the disclosure to the embodiment or embodiments described herein. On the contrary, the intent is to cover all alternatives, modifications, and equivalents.

Disclosed herein are systems and methods for manufacturing a surgical bone implant. Also disclosed are systems and methods for surgical bone implants. More specifically, the invention relates to systems and methods for manufacturing a surgical bone implant with improved physical stability relative to prior art surgical bone implants and spinal implants. As noted above, surgical bone implants may have less than desirable physical stability, particularly when such implants are made of bone fragments or pulverized bone and subjected to dehydration and/or rehydration processes.

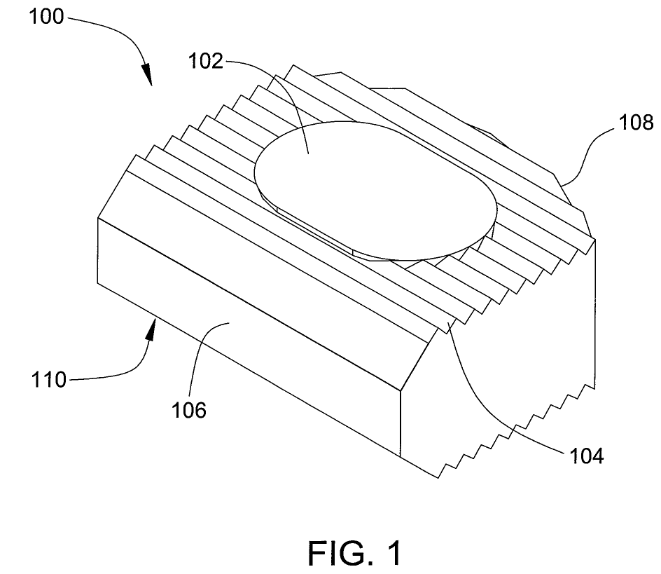

Reference is now made to FIG. 1, which illustrates a surgical bone implant 100 constructed in accordance with the invention. The depicted implant has an outer fragment 104 with serrated dorsal and ventral 110 sides. The noted serrated sides assist the implants insertion and securing between adjacent vertebral bodies during a lumbar interbody fusion procedure or other surgical spinal procedure requiring such an implant. In the depicted embodiment, the outer fragment 104 is machined from a cortical bone fragment or pulverized cortical bone fragments formed into an appropriately shaped outer fragment 104 with serrated dorsal and ventral sides. The depicted surgical bone implant 100 also has an inner fragment 102 designed to span the vertical height of the outer fragment 104. In the depicted embodiment 100, the inner fragment 102, unlike the cortical outer fragment 104, is constructed from cancellous bone tissue. The inner fragment 102 can be machined from a cancellous bone fragment or pulverized cancellous bone particles and then machined to form a shape corresponding to the inner fragment 102.

As noted above, a deficiency of spinal implants constructed in accordance with the prior art is a physical instability or weakness of the implant, particularly when subjected dehydration, hydration and sterilization procedures that are often employed as a part of the storage and transportation process after manufacture. For example, when dehydrated as a part of a storage and sterilization process, an implant may suffer from chipping or warping. Another deficiency of prior art implants is improper sizing resulting from warping during hydration or dehydration of a spinal implant. Similarly, when an implant is rehydrated for used by a surgeon similar effects may result from the rehydration process. For the depicted spinal implant 100, the rehydration process may include bathing the spinal implant 100 in an isotonic solution for a specified amount of time. An isotonic solution for humans can include a 0.9 weight percent salt aqueous solution (9 g/L salt). This is a solution that is commonly known as saline.

A rehydrated spinal implant 100 may suffer from chipping or warping during or after the rehydration process if constructed in accordance with the prior art. This can occur because prior art spinal implants expand or shrink in an unpredictable manner when subjected to hydration or dehydration. This unpredictability of prior art spinal implants occurs because individual bone fragments machined to form an implant may expand or shrink in different directions or at different rates, lowering the structural stability of the implant. Furthermore, in a prior art design, the inner fragment 102 and outer fragment 104 may expand or contract at different rates and/or in different directions, potentially causing the inner and outer fragments to exert forces on one another that may cause warping and/or structural breakdown of the implant.

To remedy these defects in the prior art, the depicted embodiment of the disclosure is constructed to allow for structural stability, particularly when undergoing dehydration, hydration and sterilization. Similarly, the embodiment is constructed to allow for achieving proper and predictable sizing, particularly in a vertical direction. In particular, either the inner fragment 102 or the outer fragment 104, or both, is machined with an osteonal direction oriented to mitigate warping or chipping under expansion or shrinking. Bone fragments expand and shrink under hydration or dehydration, respectively, in a direction predominantly perpendicular to the osteonal direction of the bone. In the depicted embodiment, the bone fragment or compacted pulverized bone material machined for the implant can be oriented such that the osteonal direction of the fragments are parallel to the direction of insertion or interlocking. Therefore, in the depicted embodiment of a spinal implant 100, the inner fragment 102 and outer fragment 104 are machined with a osteonal direction parallel to the direction of insertion of inner fragment 102 into outer fragment 104, or in a vertical direction.

Therefore, in the depicted spinal implant 100, the osteonal direction of the bone fragments used in both fragments 102, 104, is in a vertical direction from the ventral surface 110 to the dorsal surface. In other words, the fragments 102, 104 are machined such that a substantially uniform osteonal direction is perpendicular to an axis extending vertically through the dorsal and ventral 110 surfaces of the implant. Such an osteonal direction will cause the implant to expand and/or shrink in a direction consistent with a plane extending horizontally through the implant. The osteonal direction orientation of the depicted implant 100 will cause the implant to expand or shrink under hydration or dehydration in a horizontal direction, or in a direction perpendicular to the osteonal direction of the fragments used to form the implant. It should be appreciated that expansion or shrinkage along a single plane under hydration or dehydration as opposed to expansion or shrinkage in varying directions and to varying degrees will lessen the structural stress that bone comprising the implant is subjected during the manufacturing, storage, and usage processes.

The outer fragment 104 can be machined as described above with bone fragments oriented in a consistent osteonal direction. Similarly, the inner fragment 102 can be machined as described above with to a size that is larger than the aperture of the outer fragment 104. The machined inner fragment 102 can then be partially dehydrated such that its size decreases and interlocks within the aperture of the outer fragment 104, which remains in its naturally hydrated state. Subsequently, the inner and outer fragments 102, 104 can be dehydrated as an assembly for storage. Because the inner fragment 102 has been partially dehydrated prior to dehydration of the implant 100 as an assembly, the two piece implant 100 receives improved structural rigidity because the outer fragment 104 will decrease in size to a greater degree relative to the inner fragment 102. This will cause the outer fragment 104 to interlock the inner fragment 102 with more physical stability relative to prior art spinal implants.

Alternatively, the outer fragment 104 may be hydrated or bathed in a solution causing it to expand prior to engagement with the inner fragment 102. For example, the outer fragment may be bathed in a water, deionized water or subjected to reverse osmosis, which causes salt in the bone to achieve substantial equilibrium with the aqueous solution. This process causes the bone to absorb water and expand. The outer fragment 104 can be hydrated in this manner until it reaches a size that will allow it to interlock with inner fragment 102. In other words, an outer fragment 104 hydrated in this manner can be interlocked with the inner fragment 102 and dehydrated as an assembly. Because the outer fragment 104 has been hydrated and has a higher water concentration than an inner fragment 102 existing in its naturally hydrated state, when the assembly is subjected to a chemical dehydration agent, the outer fragment 104 will dehydrate and reduce in size to a greater degree relative to the inner fragment 102. This will cause outer fragment 104 to interlock the inner fragment 102 with more physical stability relative to prior art spinal implants.

It should be appreciated that many spinal implants are constructed from at least two parts as they often contain at least two bone types. In the depicted embodiment, the spinal implant contains two types of bone, cancellous and cortical, but it should be appreciated that the implant may be constructed with more or fewer bone types depending on the application and location of insertion during a surgical procedure. It should further be appreciated that while an implant constructed in accordance with the disclosure is dehydrated for storage and sterilization, the implant should be rehydrated for use by a surgeon. An implant may be stored in an evacuated storage compartment or vacuum-sealed packaging. Accordingly, an isotonic solution, saline, blood, bone marrow disparate, platelet rich plasma, sterile water, growth factors, an osteoninductive solution, an osteongenic solution or other agents utilized in the art for hydration of a bone implant may be injected or introduced into the evacuated storage compartment for rehydration of the implant. It should be appreciated and injection of a rehydration agent into an evacuated storage compartment will also speed the hydration process, as liquid is drawn into the implant tissue more rapidly relative to a non-evacuated storage compartment because of the atmospheric pressure difference.

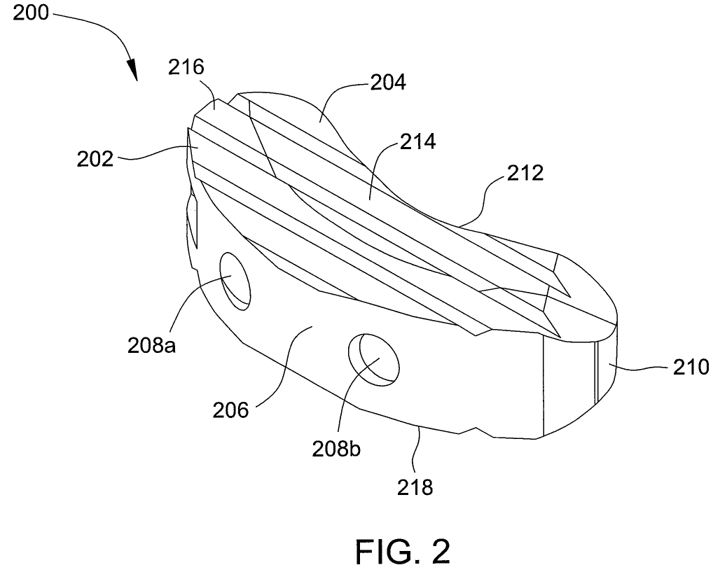

Reference is now made to FIG. 2, which depicts a spinal implant 200 of a varied design relative to the embodiment of FIG. 1 but constructed in accordance with the same above described principles. The implant 200 includes an anterior fragment 202, a posterior fragment 204 as well as pins 208a, 208b configured to interlock with apertures that span both anterior and posterior fragments 202, 204. The implant 200 also include serrated dorsal and ventral surfaces 216 and 218. The fragments utilized for spinal implant 200 include cortical bone fragments or pulverized cortical bone material. Cortical bone harvested from a single source machined to an appropriate shape may also be used for spinal implant 200. The applications of such an implant should be appreciated by a person of ordinary skill in the art, as such an implant is generally positioned with an anteriorly approach to a patient's spine.

As noted above, bone fragments 202, 204, 208a, and 208b can be formed and machined with osteonal direction configured to reduce the effects of expansion and shrinkage that may potentially warp or chip the implant 200. In the depicted embodiment, pins 208a and 208b can be machined such that the osteonal direction of the pins runs longitudinally along the cylindrical shape of the pins 208a, 208b. In other words, the pins 208a, 208b can be machined such that the osteonal direction of the bone comprising the pins 208, 208b is parallel to the direction of insertion into the apertures of fragments 202, 204. As noted above, such orientation of the osteonal direction of the bone comprising the implant 200 can assist in the structural stability of the implant 200 that may be subjected to dehydration and hydration. Pins 208a, 208b machined in such a manner will cause the pins 208a, 208b to expand or shrink predominantly in a radial direction relative to the cylindrical shape of the pins. Similarly, preferably, fragments 202, 204 are machined such that the osteonal direction is substantially the same as pins 208a, 208b. As noted above, bone fragments will predominantly expand or shrink in a direction perpendicular to a substantially consistent osteonal direction, resulting in a more predictable size of the implant as well as resistance to warping or chipping of the implant during dehydration, sterilization and rehydration due to warping.

Similar to the above noted embodiment of FIG. 1, depicted spinal implant 200 can also be constructed with a staggered dehydration technique that also improves the structural stability of an implant that has been dehydrated for storage and/or transport as well as assist in achieving proper and predictable sizing of the implant 200. Pins 208a, 208b can be machined as described above with compacted bone fragments oriented in a consistent osteonal direction to a size that is larger than the apertures of fragments 202, 204. The machined pins 208a, 208b can then be partially dehydrated such that their size decreases and interlocks within the apertures of outer fragments 202, 204, which remains hydrated. Subsequently, fragments 202, 204 and pins 208a, 208b can be dehydrated as an assembly for sterilization and storage. Because the pins 208a, 208b have been partially dehydrated prior to dehydration of the implant 200 as an assembly, the implant 200 receives improved structural rigidity because fragments 202, 204 will decrease in size to a greater degree relative to the inner pins 208a, 208b. This will cause the outer fragments 202, 204 to interlock the pins 208a, 208b with more physical stability relative to prior art spinal implants.

Alternatively, the outer fragments 202, 204 may be hydrated or bathed in a solution causing it to expand prior to engagement with pins 208a, 208b. For example, the outer fragment may be bathed in a water, deionized water or subjected to reverse osmosis, which causes salt in the bone to achieve substantial equilibrium with the aqueous solution. This process causes the bone to absorb water and expand. Subsequently, outer fragments 202, 204 hydrated in this manner can be interlocked with pins 208a, 208b and dehydrated as an assembly. Because outer fragments 202, 204 have been hydrated and have a higher water concentration than pins 208a, 208b existing in their naturally hydrated state, when the assembly is subjected to a chemical dehydration agent, outer fragments 202, 204 will dehydrate and reduce in size to a greater degree relative to pins 208a, 208b. This will cause outer fragments 202, 204 to interlock with pins 208a, 208b with more physical stability relative to prior art spinal implants.

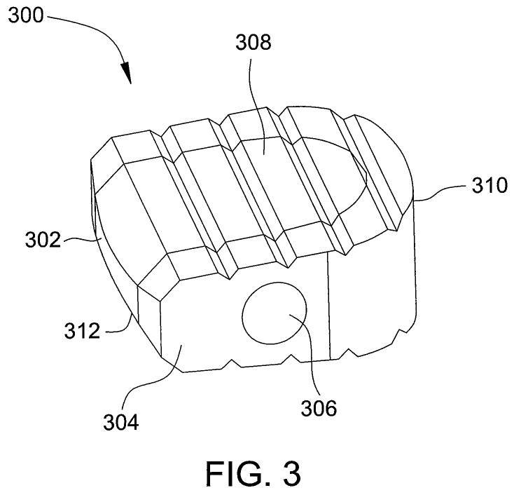

Reference is now made to FIG. 3, which depicts a spinal implant 300 of a varied design relative to the above disclosed but constructed in accordance with the same above described principles. The implant 300 includes an outer fragment 304, an inner fragment 302 as well as pin 306 configured to interlock with an aperture spanning both inner and outer fragments 302, 304. The implant 300 also include a serrated dorsal and ventral surfaces 308 and 312. Outer fragment 304 is comprised of cortical bone fragments, while inner fragment 302 is comprised of cancellous bone. Pin 306 is comprised of cortical bone and is designed to assist in the securing of inner and outer fragments 302, 304.

As noted above, bone fragments 302, 304 and pin 306 can be formed and machined with osteonal direction configured to reduce the effects of expansion and shrinkage that may potentially warp or chip the implant 300. In the depicted embodiment, pin 306 can be machined such that the osteonal direction of the pin runs longitudinally along the cylindrical shape of the pin 306. In other words, the pin 306 can be machined such that the osteonal direction of the bone comprising the pin 306 is parallel to the direction of insertion into the apertures of fragments 302, 304. As noted above, such orientation of the osteonal direction of the bone comprising the implant 300 can assist in the structural stability of the implant 300 that may be subjected to dehydration and hydration. Pin 306 is machined in such a manner will cause the pin 306 to expand or shrink predominantly in a radial direction relative to the cylindrical shape of the pin. Similarly, preferably, fragments 302, 304 are machined such that the osteonal direction is substantially the same as pins 306. As noted above, bone fragments will predominantly expand or shrink in a direction perpendicular to a substantially consistent osteonal direction, resulting in a more predictable size of the implant as well as resistance to warping or chipping of the implant during dehydration, sterilization and rehydration due to warping.

Similar to the above noted embodiments, depicted spinal implant 300 is also constructed with a staggered dehydration technique that also improves the structural stability of an implant that has been dehydrated for storage and/or transport as well as assist in achieving proper and predictable sizing of the implant 300. Pin 306 can be machined as described above with compacted bone fragments oriented in a consistent osteonal direction to a size that is larger than the apertures of fragments 302, 304. The machined pin 306 can then be partially dehydrated such that its size decreases and interlocks within the apertures of fragments 302, 304, which remain hydrated. Subsequently, fragments 302, 304 and pin 306 can be dehydrated as an assembly for sterilization and storage. Because the pin 306 has been partially dehydrated prior to dehydration of the implant 300 as an assembly, the implant 300 receives improved structural rigidity because fragments 302, 304 will decrease in size to a greater degree relative to the pin 306. This will cause the outer fragments 302, 304 to engage the pin 306 with more physical stability relative to prior art spinal implants.

Alternatively, the outer fragments 302, 304 may be hydrated or bathed in a solution causing it to expand prior to engagement with pin 306. For example, the outer fragment may be bathed in a water, deionized water or subjected to reverse osmosis, which causes salt in the bone to achieve substantial equilibrium with the aqueous solution. This process causes the bone to absorb water and expand. Subsequently, outer fragments 302, 304 hydrated in this manner can be interlocked with pin 306 and dehydrated as an assembly. Because outer fragments 302, 304 have been hydrated and have a higher water concentration than pin 306 existing in its naturally hydrated state, when the assembly is subjected to a chemical dehydration agent, outer fragments 302, 304 will dehydrate and reduce in size to a greater degree relative to pin 306. This will cause outer fragments 302, 304 to interlock with pin 306 with more physical stability relative to prior art spinal implants.

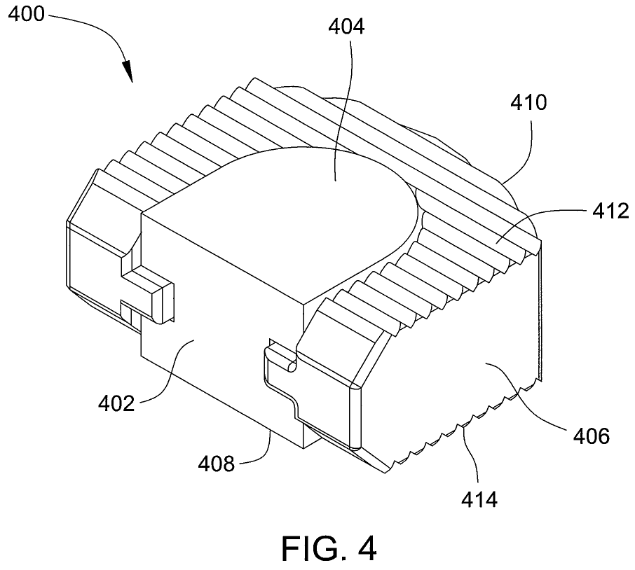

Reference is now made to FIG. 4, which depicts a spinal implant 400 of a varied design relative to the above disclosed but constructed in accordance with the same above described principles. The implant 400 includes an outer fragment 406, and an inner fragment 404 configured to interlock outer fragment 406. The implant 400 also includes an outer fragment 406 with serrated dorsal and ventral surfaces. Outer fragment 406 is comprised of cortical bone, while inner fragment 404 is comprised of cancellous bone.

As noted above, bone fragments that are machined to form fragments 404, 406 can be formed and machined with an osteonal direction configured to reduce the effects of expansion and shrinkage that may potentially warp or chip the implant 400. In the depicted implant 400, the inner fragment 404 is machined with a osteonal direction oriented in the direction of insertion into the outer fragment 406. In other words, the osteonal direction is in a direction running from anterior end 402 to posterior end 410. Outer fragment 406 may be machined with an osteonal direction parallel to the osteonal direction of the bone comprising the inner fragment 404. As further noted above, bone fragments will predominantly expand or shrink in a direction perpendicular to a substantially consistent osteonal direction, resulting in a more predictable size in the vertical dimension of the implant as well as resistance to warping or chipping of the implant during dehydration, sterilization and rehydration.

Similar to the above noted embodiments, depicted spinal implant 400 is also constructed with a staggered dehydration technique that also improves the structural stability of an implant that has been dehydrated for storage and/or transport or rehydrated for use. It may also assist in achieving proper and predictable sizing of the implant 400 by limiting warping or unpredictable expansion or shrinkage. Inner fragment 404 can then be partially dehydrated such that its size decreases and interlocks within inner fragment 406, which remains in its naturally hydrated state. Subsequently, fragments 404, 406 can be dehydrated as an assembly for sterilization and storage. Because the fragment 404 has been partially dehydrated prior to dehydration of the implant 400 as an assembly, the implant 400 receives improved structural rigidity because fragment 406 will decrease in size to a greater degree relative to fragment 404. This will cause the outer fragments 404 and 406 to interlock with more physical stability relative to prior art spinal implants.

Alternatively, the outer fragment 406 may be hydrated or bathed in a solution causing it to expand prior to engagement with inner fragment 404. For example, the outer fragment may be bathed in a water, deionized water or subjected to reverse osmosis, which causes salt in the bone to achieve substantial equilibrium with the aqueous solution. This process causes the bone to absorb water and expand. Subsequently, outer fragment 406 hydrated in this manner can be interlocked with inner fragment 404 and dehydrated as an assembly. Because outer fragment 406 has been hydrated and have a higher water concentration than inner fragment 404 existing in its naturally hydrated state, when the assembly is subjected to a chemical dehydration agent, outer fragment 406 will dehydrate and reduce in size to a greater degree relative to inner fragment 404. This will cause outer fragment 406 to interlock with inner fragment 404 with more physical stability relative to prior art spinal implants.

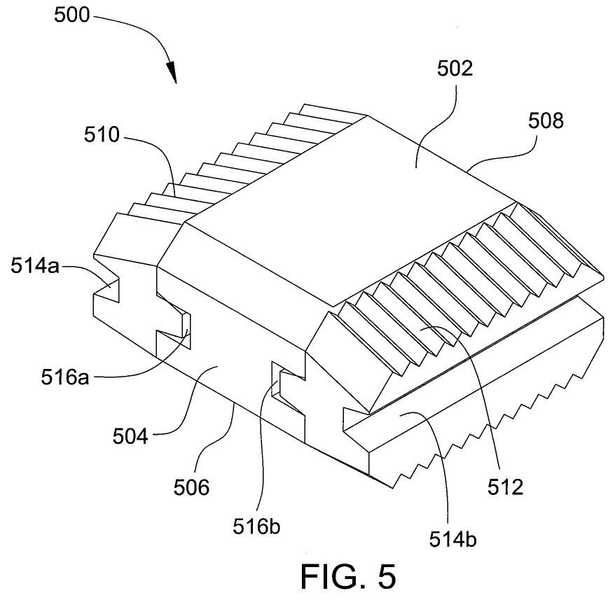

Reference is now made to FIG. 5, which depicts a spinal implant 500 of a varied design relative to the above disclosed embodiments but constructed in accordance with the same above described principles. The implant 500 includes outer fragments 510, 512, and an inner fragment 502 configured to interlock outer fragments 510, 512. The implant 500 also includes outer fragments 510, 512 with serrated dorsal and ventral surfaces. Outer fragments 510, 512 are comprised of cortical bone, while inner fragment 504 is comprised of cancellous bone.

As noted above, bone fragments that are machined to form fragments 510, 512, and 502 can be formed and machined with an osteonal direction configured to reduce the effects of expansion and shrinkage that may potentially warp or chip the implant 500. In the depicted implant the inner fragment 502 can be machined such that the osteonal direction of the bone comprising the fragment is in the horizontal direction. Or, in other words, the osteonal direction is oriented towards the point of insertion in a plane beginning at interlocking notch 516a traveling towards interlocking notch 516b. As noted above, bone fragments will predominantly expand or shrink in a direction perpendicular to a substantially consistent osteonal direction, resulting in a more predictable sizing of the implant as well as resistance to warping or chipping of the implant during dehydration, sterilization and rehydration.

Similar to the above noted embodiments, depicted spinal implant 500 is also constructed with a staggered dehydration technique that also improves the structural stability of an implant that has been dehydrated for storage and/or transport or rehydrated for use. It may also assist in achieving proper and predictable sizing of the implant 500 by limiting warping or unpredictable expansion or shrinkage. Inner fragment 502 can be partially dehydrated such that its size decreases and interlocks within outer fragments 510, 512, which remains in its naturally hydrated state. Subsequently, fragments 510, 512, and 504 can be dehydrated as an assembly for sterilization and storage. Because the fragment 504 has been partially dehydrated prior to dehydration of the implant 500 as an assembly, the implant 500 receives improved structural rigidity because fragments 510, 512 will decrease in size to a greater degree relative to fragment 502. This will cause the outer fragments 510, 512 to interlock with inner fragment 502 more physical stability relative to prior art spinal implants.

Alternatively, the outer fragments 510, 512 may be hydrated or bathed in a solution causing it to expand prior to engagement with inner fragment 502. For example, the outer fragment may be bathed in water, deionized water or subjected to reverse osmosis, which causes salt in the bone to achieve substantial equilibrium with the aqueous solution. This process causes the bone to absorb water and expand. Subsequently, outer fragments 510, 512 hydrated in this manner can be interlocked with inner fragment 502 and dehydrated as an assembly. Because outer fragments 510, 512 has been hydrated and have a higher water concentration than inner fragment 502 existing in its naturally hydrated state, when the assembly is subjected to a chemical dehydration agent, outer fragments 510, 512 will dehydrate and reduce in size to a greater degree relative to inner fragment 502. This will cause outer fragments 510, 512 to interlock with inner fragment 502 with more physical stability relative to prior art spinal implants.

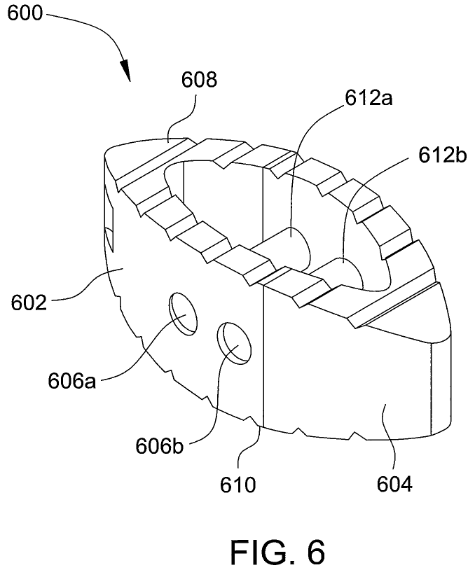

Reference is now made to FIG. 6, which depicts a spinal implant 600 of a varied design relative to the above disclosed embodiments but constructed in accordance with the same above described principles. The implant 600 includes interlocking outer fragments 602, 604, and pins 612a, 612b configured to interlock outer fragments 602, 604 by extending through apertures 606a, 606b. The implant 600 also includes outer fragments 602, 604 with serrated dorsal 608 and ventral surfaces 610. Interlocking outer fragments 602, 604 are comprised of cortical bone, as are pins 612a, 612b. It should be appreciated that implant 600 can be used for spinal procedures including but not limited to transforaminal lumbar interbody fusion.

As noted above, fragments 602, 604, and pins 612a, 612b can be formed and machined with an osteonal direction configured to reduce the effects of expansion and shrinkage that may potentially warp or chip the implant 600. In the depicted embodiment, pins 612a and 612b can be machined such that the osteonal direction of the pins runs longitudinally along the cylindrical shape of the pins 612a, 612b. In other words, the pins 612a, 612b can be machined such that the osteonal direction of the bone comprising the pins 612, 612b is parallel to the direction of insertion into the apertures of fragments 602, 604. As noted above, such orientation of the osteonal direction of the bone comprising the implant 600 can assist in the structural stability of the implant 600 that may be subjected to dehydration and hydration. Pins 612a, 612b machined in such a manner will cause the pins 612a, 612b to expand or shrink predominantly in a radial direction relative to the cylindrical shape of the pins. As noted above, bone fragments will predominantly expand or shrink in a direction perpendicular to a substantially consistent osteonal direction, resulting in a more predictable size of the implant as well as resistance to warping or chipping of the implant during dehydration, sterilization and hydration due to warping.

Similar to the above noted embodiments, depicted spinal implant 600 is also constructed with a staggered dehydration technique that also improves the structural stability of an implant that has been dehydrated for storage and/or transport or rehydrated for use. It may also assist in achieving proper and predictable sizing of the implant 600 by limiting warping or unpredictable expansion or shrinkage. Pins 612a, 612b can be machined as described above with compacted bone fragments oriented in a consistent osteonal direction to a size that is larger than the apertures of fragments 602, 604. The machined pins 612a, 612b can then be partially dehydrated such that their size decreases and interlocks within the apertures of outer fragments 602, 604, which remains hydrated. Subsequently, fragments 602, 604 and pins 612a, 612b can be dehydrated as an assembly for sterilization and storage. Because the pins 612a, 612b have been partially dehydrated prior to dehydration of the implant 600 as an assembly, the implant 600 receives improved structural rigidity because fragments 602, 604 will decrease in size to a greater degree relative to the pins 612a, 612b. This will cause the outer fragments 602, 604 to interlock the pins 612a, 612b with more physical stability relative to prior art spinal implants.

Alternatively, the outer fragments 602, 604 may be hydrated or bathed in a solution causing it to expand prior to engagement with pins 612a, 612b. For example, the outer fragment may be bathed in a water, deionized water or subjected to reverse osmosis, which causes salt in the bone to achieve substantial equilibrium with the aqueous solution. This process causes the bone to absorb water and expand. Subsequently, outer fragments 602, 604 hydrated in this manner can be interlocked with pins 612a, 612b and dehydrated as an assembly. Because outer fragments 602, 604 have been hydrated and have a higher water concentration than pins 612a, 612b existing in their naturally hydrated state, when the assembly is subjected to a chemical dehydration agent, outer fragments 602, 604 will dehydrate and reduce in size to a greater degree relative to pins 612a, 612b. This will cause outer fragments 602, 604 to interlock with pins 612a, 612b with more physical stability relative to prior art spinal implants.

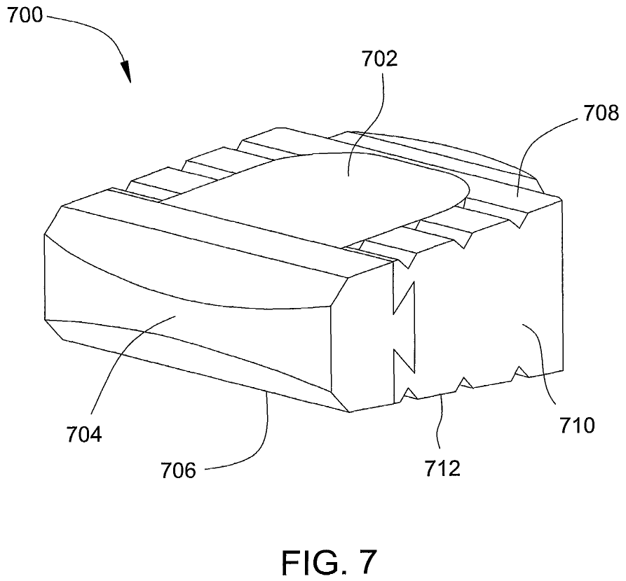

Reference is now made to FIG. 7, which depicts a spinal implant 700 of a varied design relative to the above disclosed embodiments but constructed in accordance with the same above described principles. The implant 700 includes interlocking outer fragments 704, 708, and inner fragment 702. The implant 700 also includes outer fragment 708 with serrated dorsal and ventral 712 surfaces. Interlocking outer fragments 704, 708 are comprised of cortical bone, whereas inner fragment 702 is comprised of cancellous bone.

As noted above, compacted bone fragments that are machined to form larger fragments 704, 708 and inner fragment 702 can be formed and machined with an osteonal direction configured to reduce the effects of expansion and shrinkage that may potentially warp or chip the implant 700. In the depicted implant the inner fragment 702 can be machined such that the osteonal direction of the bone comprising the fragment is in the horizontal direction. Or, in other words, the osteonal direction is oriented in a plane beginning at proximal end 704 and spanning to the distal end of the implant 700. As noted above, an osteonal direction oriented toward the point of interlocking between a male and female portion of a joint connecting multiple bone fragments forming the implant 700 can assist in the structural stability of the implant 700 that may be subjected to dehydration and hydration. It may also assist in achieving proper sizing because of resistance to warping during dehydration and/or hydration. As noted above, bone fragments will predominantly expand or shrink in a direction perpendicular to a substantially consistent osteonal direction, resulting in a more predictable sizing of the implant because resistance to warping or chipping of the implant during dehydration, sterilization and rehydration is promoted.

Similar to the above noted embodiments, depicted spinal implant 700 is also constructed with a staggered dehydration technique that also improves the structural stability of an implant that has been dehydrated for storage and/or transport or rehydrated for use. It may also assist in achieving proper and predictable sizing of the implant 700 by limiting warping or unpredictable expansion or shrinkage. Inner fragment 702 can then be partially dehydrated such that its size decreases and interlocks within fragments 704, 708, which remain in their naturally hydrated state. Subsequently, fragments 704, 708, and inner fragment 702 can be dehydrated as an assembly for sterilization and storage. Because inner fragment 702 has been partially dehydrated prior to dehydration of the implant 700 as an assembly, the implant 700 receives improved structural rigidity because fragments 704, 708 will decrease in size to a greater degree relative to inner fragment 702. This will cause the fragments 704, 708 to interlock with inner fragment 702 with more physical stability relative to prior art spinal implants.

Alternatively, the outer fragments 704, 708 may be hydrated or bathed in a solution causing it to expand prior to engagement with inner fragment 702. For example, the outer fragment may be bathed in water, deionized water or subjected to reverse osmosis, which causes salt in the bone to achieve substantial equilibrium with the aqueous solution. This process causes the bone to absorb water and expand. Subsequently, outer fragments 704, 708 hydrated in this manner can be interlocked with inner fragment 702 and dehydrated as an assembly. Because outer fragments 704, 708 has been hydrated and have a higher water concentration than inner fragment 702 existing in its naturally hydrated state, when the assembly is subjected to a chemical dehydration agent, outer fragments 704, 708 will dehydrate and reduce in size to a greater degree relative to inner fragment 702. This will cause outer fragments 704, 708 to interlock with inner fragment 702 with more physical stability relative to prior art spinal implants.

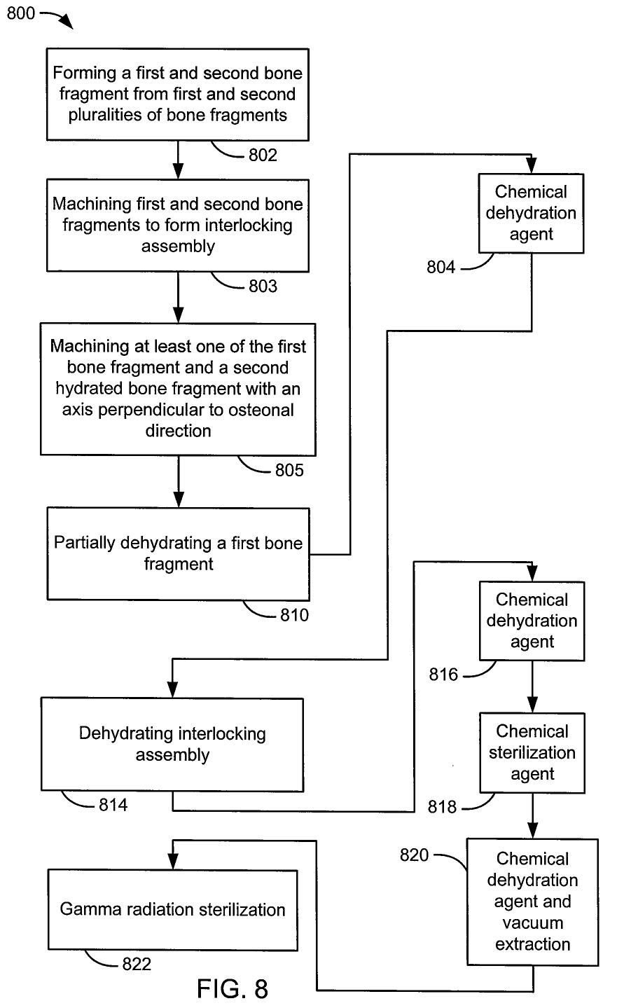

Reference is now made to FIG. 8, which discloses a method 800 in accordance with the disclosure. FIG. 8 discloses steps undertaken to manufacture a surgical bone implant in accordance with the disclosure. The method 800 begins at step 802, where at least a first and second bone fragments are formed from a first plurality and second plurality of bone fragments. It should be appreciated that a greater number of bone fragments may be formed if a spinal implant application requires more than two bone fragments. It should also be appreciated that the first and second bone fragments may be formed from cancellous, cortical, or corticocancellous compacted bone fragments.

In step 803, the first and second bone fragments are then machined to form an interlocking assembly. As noted above, the second of the interlocking bone fragments may be machined with an aperture or a female interlocking shape and the first of the bone fragments may be machined to form a pin or other shape designed to interlock within the aperture or a male interlocking shape. However, the first bone fragment can be machined to a size larger than the aperture or interlocking shape of the first bone fragment such that it may fit within such an aperture after undergoing partial dehydration and resulting shrinking in a subsequent step of the embodiment.

In step 805, at least one or all of the formed bone fragments are machined with at least one axis perpendicular to an osteonal direction. As noted above in reference to previous embodiments of the disclosure, the expansion or shrinkage of multiple bone fragments connected by a joint that are also formed from bone wherein the bone fragments are configured with an osteonal direction parallel to a direction of insertion occurs predominantly in a direction perpendicular to the osteonal direction. In this way, expansion or shrinkage under hydration or dehydration can be predictable and an implant configured such that expansion or shrinkage does not cause warping or chipping of the implant. Furthermore, such orientation of the bone material in an implant increases the physical stability of the implant caused by expansion or shrinkage in different direction or to different degrees. In step 810, at least one of the bone fragments is partially dehydrated. As noted above in reference to step 803, the first bone fragment can be machined to a size larger than an aperture or interlocking shape of the second bone fragment, as the first bone fragment will reduce in size under dehydration.

Dehydration can be accomplished in various ways that should be appreciated by a person of ordinary skill in the art. In the depicted embodiment, dehydration of the first bone fragment is accomplished in step 804. In step 804, the first bone fragment is exposed to a chemical dehydration agent. The chemical dehydration agent can include but is not limited to an acetone, an organic solvent, a detergent, and an oxidizing agent. It should further be appreciated that dehydration may be accomplished in other ways known to a person of ordinary skill in the art. It should further be appreciated that lipids existing in the bone fragments will be removed by exposing the bone fragment to a dehydration agent such as an acetone bath. It should also be noted an ultrasonic acetone bath may also be employed in step 804 as part of the lipid removal process.

In step 814, the first bone fragment and second bone fragment are interlocked and dehydrated as an assembly. The dehydration process step 816 for the interlocking assembly are similar to the above reference step 804. In addition, in step 818, the assembly is exposed to a hydrogen peroxide bath, which removes soluble proteins as well as destroys various viruses and bacterial spores that may exist within the bone fragment. In step 820, a second exposure to a chemical dehydration agent as well as vacuum extraction is employed to finalize the dehydration process. As noted above, this dehydration step results in a surgical bone implant possessing greater physical stability and more predictable sizing relative to prior art bone implant manufacturing processes, as the implant is less susceptible to warping, chipping, and it also ensures that a multi-part implant including interlocking bone fragments remains connected in a single assembly. In step 822, sterilization of the bone implant is completed by exposing the implant to low dose gamma radiation. Step 822 ensures the elimination of microbial contamination remaining in the implant.

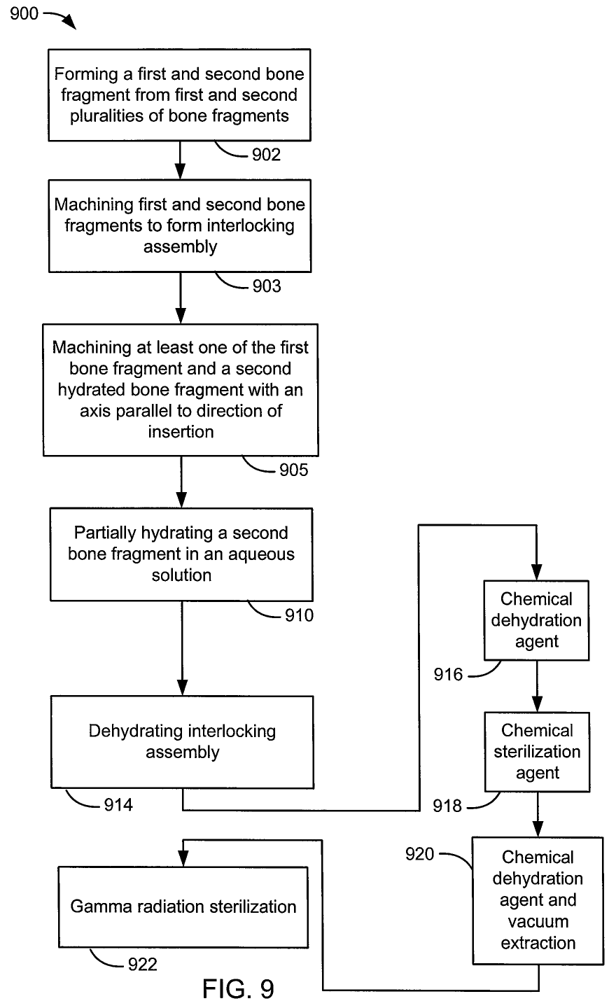

Reference is now made to FIG. 9, which illustrates an alternative method embodiment of the disclosure. FIG. 9 discloses steps undertaken to manufacture a surgical bone implant in accordance with the disclosure. The method 900 begins at step 902, where at least a first and second bone fragments are formed or harvested for use in a surgical bone implant. It should be appreciated that a greater number of bone fragments may be formed if a spinal implant application requires more than two bone fragments. It should also be appreciated that the first and second bone fragments may be formed from cancellous, cortical, or corticocancellous bone fragments.

In step 903, the first and second bone fragments are then machined to form an interlocking assembly. As noted above, the second of the interlocking bone fragments may be machined with an aperture or a female interlocking shape and the first of the bone fragments may be machined to form a pin or other shape designed to interlock within the aperture or a male interlocking shape.

In step 905, at least one or all of the formed bone fragments are machined with an osteonal direction parallel to the direction of insertion of the male fragment into the female fragment as they are to be joined as an assembly. As noted above in reference to previous embodiments of the disclosure, the expansion or shrinkage of multiple bone fragments connected by a joint that are also formed from bone wherein the bone fragments are configured with an osteonal direction parallel to a direction of insertion occurs predominantly in a direction perpendicular to the osteonal direction. In this way, expansion or shrinkage under hydration or dehydration can be predictable and an implant configured such that expansion or shrinkage does not cause warping or chipping of the implant. Furthermore, such orientation of the bone material in an implant increases the physical stability of the implant caused by expansion or shrinkage in different direction or to different degrees.

In step 910, at least one of the bone fragments is partially hydrated in an aqueous solution. Hydration can be accomplished in various ways that should be appreciated by a person of ordinary skill in the art. For example, the second bone fragment may be bathed in water, deionized water or subjected to reverse osmosis, which causes salt in the bone to achieve substantial equilibrium with the aqueous solution. This process causes the bone to absorb water and expand. Subsequently, the second bone fragment hydrated in this manner can be interlocked with the first bone fragment in step 914 and dehydrated as an assembly. Because the second bone fragment has been hydrated and has a higher water concentration than the first bone fragment in its naturally hydrated state, when the assembly is subjected to a chemical dehydration agent, the second hydrated bone fragment will dehydrate and reduce in size to a greater degree relative to the first bone fragment. This will cause the second bone fragment to interlock with the first bone fragment with more physical stability relative to prior art spinal implants.

Accordingly, in step 914, the first bone fragment and second bone fragment are interlocked and dehydrated as an assembly. The dehydration process step 916 for the interlocking assembly are similar to the above referenced step 804. In addition, in step 918, the assembly is exposed to a hydrogen peroxide bath, which removes soluble proteins as well as destroys various viruses and bacterial spores that may exist within the bone fragment. In step 920, a second exposure to a chemical dehydration agent as well as vacuum extraction is employed to finalize the dehydration process. As noted above, this dehydration step results in a surgical bone implant possessing greater physical stability and more predictable sizing relative to prior art bone implant manufacturing processes, as the implant is less susceptible to warping, chipping, and it also ensures that a multi-part implant including interlocking bone fragments remains connected in a single assembly. In step 922, sterilization of the bone implant is completed by exposing the implant to low dose gamma radiation. Step 922 ensures the elimination of microbial contamination remaining in the implant.

It should be emphasized that the above-described embodiments of the present invention, particularly, any “preferred” embodiments, are merely possible examples of implementations, set forth for a clear understanding of the principles of the invention. Many variations and modifications may be made to the above-described embodiment(s) of the invention without departing substantially from the spirit and principles of the invention. All such modifications and variations are intended to be included herein within the scope of this disclosure and the present invention and protected by the following claims.

In light of the foregoing, at least the following is claimed:

1. A method of manufacturing a surgical bone implant, comprising the steps of:
shaping a first bone fragment for engagement with a second bone fragment, the first bone fragment configured to interlock with the second bone fragment in at least one joint, the first bone fragment machined as a male portion of the at least one joint and the second bone fragment machined as a female portion of the at least one joint,
shaping at least one of the first bone fragment and the second bone fragment with an osteonal direction parallel to a direction of insertion with the second bone fragment,
at least partially dehydrating the first bone fragment,
interlocking the first bone fragment with the second bone fragment such that the male portion is at least partially inserted into the female portion, and
at least partially dehydrating the interlocking first bone fragment and second bone fragment.

2. The method of claim 1, wherein the step of at least partially dehydrating the first bone fragment and the step of at least partially dehydrating the interlocking first bone fragment and second bone fragment further comprises the steps of:
placing at least one of the first bone fragment and the second bone fragment in a chemical dehydration solvent.

3. The method of claim 1, wherein the chemical dehydration solvent is chosen from: acetone, an organic solvent, a detergent, and an oxidizing agent.

4. The method of claim 1, further comprising the steps of:
sterilizing the interlocking first bone fragment and second bone fragment by exposing the interlocking first bone fragment and second bone fragment to gamma radiation.

5. The method of claim 1, further comprising the steps of:
packaging the interlocked first and second bone fragments in an evacuated storage compartment.

6. The method of claim 5, further comprising the steps of:
injecting a hydrating solution into the evacuated storage compartment.

7. The method of claim 6, wherein the hydrating solution is chosen from: an isotonic solution, saline, blood, bone marrow disparate, platelet rich plasma, sterile water, growth factors, osteoninductive solution, and osteongenic solution.

8. The method of claim 1, wherein:
the first bone fragment is machined into a pin shape and the second bone fragment is machined with an aperture with a diameter substantially similar to a diameter of the pin shape of the first bone fragment, and the second bone fragment is configured to accept and substantially surround the first bone fragment.

9. The method of claim 1, wherein:
the first bone fragment is machined with a first surface having a convex shape, and the second bone fragment is machined with a second surface having a concave shape, the first surface and the second surface configured to engage each other.

10. The method of claim 1, wherein:
the first bone fragment is machined with a first surface having a outward protruding shape, and the second bone fragment is machined with a second surface having a substantially matching inward protruding shape such that the first surface and the second surface interlock the first bone fragment and the second bone fragment.

11. The method of claim 10, wherein the outward protruding shape is a substantially rectangular outword protruding shape.

12. The method of claim 1, wherein:
the first bone fragment is machined from at least one chosen from: cancellous bone and cortical bone; and
the second bone fragment is machined from at least one chosen from: cancellous bone and cortical bone.

13. The method of claim 1, further comprising the steps of:
machining the first bone fragment and second bone fragment to form an interlocking structure, the interlocking structure configured in the shape of a spinal interbody implant.

14. A method of manufacturing a surgical bone implant, comprising the steps of:
shaping a first bone fragment for engagement with a second bone fragment, the first bone fragment configured to interlock with the second bone fragment in at least one joint, the first bone fragment machined as a male portion of the at least one joint and the second bone fragment machined as a female portion of the at least one joint,
shaping at least one of the first bone fragment and the second bone fragment with an osteonal direction parallel to a direction of insertion with the second bone fragment,
at least partially hydrating the second bone fragment,
interlocking the first bone fragment with the second bone fragment such that the male portion is at least partially inserted into the female portion, and
at least partially dehydrating the interlocking first bone fragment and second bone fragment.

15. The method of claim 14, wherein the step of at least partially dehydrating the interlocking first bone fragment and second bone fragment further comprises the steps of:
placing the interlocking first bone fragment and second bone fragment in a chemical dehydration solvent.

16. The method of claim 14, wherein the chemical dehydration solvent is chosen from: an organic solvent, a detergent, and an oxidizing agent.

17. The method of claim 16, further comprising the steps of:
sterilizing the interlocking first bone fragment and second bone fragment by exposing the interlocking first bone fragment and second bone fragment to gamma radiation

18. The method of claim 14, further comprising the steps of:
storing the interlocked first and second bone fragments in an evacuated storage compartment.

19. The method of claim 18, further comprising the steps of:
injecting a hydrating solution into the evacuated storage compartment.

20. The method of claim 14, wherein the step of hydrating further comprises:
subjecting the second bone fragment to at least one chosen from: deionized water bath, water bath, and reverse osmosis.

21. The method of claim 14, wherein:
the first bone fragment is machined into a pin shape and the second bone fragment is machined with an aperture with a diameter substantially similar to a diameter of the pin shape of first bone fragment, and the second bone fragment is configured to accept and substantially surround the first bone fragment.

22. The method of claim 14, wherein:
the first bone fragment is machined with a first surface having a outward protruding shape, and the second bone fragment is machined with a second surface having a substantially matching inward protruding shape such that the first surface and the second surface interlock the first bone fragment and the second bone fragment.

23. The method of claim 22, wherein the outward protruding shape is a substantially rectangular outword protruding shape.

24. The method of claim 14, wherein:
the first bone fragment is machined from at least one chosen from: cancellous bone and cortical bone; and
the second bone fragment is machined from at least one chosen from: cancellous bone and cortical bone.

25. The method of claim 14, further comprising the steps of:
machining the first bone fragment and second bone fragment to form an interlocking structure, the interlocking structure configured in the shape of a spinal interbody implant.

26. A surgical bone implant, comprising:
a first bone fragment machined for interlocking with a second bone fragment; wherein,
the first bone fragment is machined to interlock with the second bone fragment in at least one joint, the first bone fragment machined as a male portion of the at least one joint and the second bone fragment machined as a female portion of the at least one joint
at least one of the first bone fragment and the second bone fragment is machined with an osteonal direction parallel to a direction of insertion with the second bone fragment; and,
the first bone fragment and the second bone fragment are configured to form an interlocking assembly by dehydrating the first bone fragment, interlocking the first bone fragment and the second bone fragment to form an assembly by at least partially inserting the male portion into the female portion, and further dehydrating the assembly.

27. The surgical bone implant of claim 26, wherein:
the first bone fragment is a plurality of bone fragments chosen from: mineralized bone fragments, demineralized bone fragments, and partially demineralized bone fragments, and
the second bone fragment is a plurality of bone fragments chosen from: mineralized bone fragments, demineralized bone fragments, and partially demineralized bone fragments.

28. The surgical bone implant of claim 26, wherein the assembly is configured in the shape of a spinal interbody implant.

29. The surgical bone implant of claim 26, wherein the first bone fragment comprises a plurality of bone fragments configured to form an interlocking assembly with the second bone fragment.

30. The surgical bone implant of claim 26, wherein:
the first bone fragment is machined from at least one chosen from: cancellous bone and cortical bone; and
the second bone fragment is machined from at least one chosen from: cancellous bone and cortical bone.
